(19) United States
(12) Patent Application Publication
(10) Pub. No.: US 2025/0229097 A1
(43) Pub. Date: Jul. 17, 2025

(54) NEUROPHYSIOLOGICAL MAGNETIC STIMULATION SYSTEM

(71) Applicants: Adam Bied, Reading, MA (US); Susan Njuguna, Reading, MA (US)

(72) Inventors: Adam Bied, Reading, MA (US); Susan Njuguna, Reading, MA (US)

(21) Appl. No.: 18/410,349

(22) Filed: Jan. 11, 2024

(51) Int. Cl. A61N 2/00

(52) U.S. Cl. CPC A61N 2/006

(57) ABSTRACT

A neurophysiological magnetic stimulation (NMS) method includes obtaining motor thresholds over one or both motor strips adhered to the patient's head or the equivalent anatomic region for treatment of non-brain, nervous system disorders. The method further includes applying magnetic stimulation coils to a therapeutic target region of the patient's brain, brainstem, spinal cord, or peripheral nervous system and applying NMS in increasing amplitudes as tolerated over the course of treatment.

### BACKGROUND OF THE INVENTION

### Field of the Invention

The present invention generally relates to a neurophysiological magnetic stimulation (NMS) system such as a system for transcranial magnetic stimulation (TMS), and more particularly to a repetitive NMS (rNMS) system that provides increasing amplitude of magnetic fields to a patient over the course of patient's treatment.

### Description of the Related Art

Transcranial magnetic stimulation (TMS) has shown diagnostic and therapeutic potential for a variety of central and peripheral nervous system (CNS, PNS) disease states, notably in the fields of neurology and mental health. TMS is a procedure that uses magnetic fields to stimulate nerve cells in a patient's brain, brainstem, spinal cord or peripheral nervous system for therapeutic purposes.

Conventional TMS systems are not optimally effective in treating psychiatric and nervous system illnesses. They frequently fail to provide a robust treatment response. Specifically, many patients undergoing conventional TMS do not respond to TMS or respond with a modest treatment improvement.

Therefore, a neurophysiological magnetic stimulation system is required that provides better response to treatment for patients with disorders of the nervous system such as mental illnesses and neurologic disorders.

### SUMMARY OF THE INVENTION

The following presents a simplified summary of the present disclosure in a simplified form as a prelude to the more detailed description that is presented herein.

The present disclosure employs language appropriate for the brain and presupposes a brain disorder as the index example though the system and method disclosed herein are equally appropriate for disorders of the brainstem, spinal cord, or peripheral nervous system in which the equivalent anatomic sites are subject to the same approach as herein described.

In accordance with embodiments of the invention, there is provided a neurophysiological magnetic stimulation (NMS) method. The method includes obtaining motor thresholds over one or both motor strips adhered to a patient's head or, if used peripherally, an equivalent nervous system region. The method further includes applying magnetic stimulation coils to a therapeutic target region of a patient's brain, brainstem, spinal cord, or peripheral nervous system region, and applying NMS pulses in increasing amplitudes as tolerated over a treatment session.

In some aspects, the method additionally includes identifying a brain, brainstem, spinal cord or peripheral nervous system disorder of a patient before obtaining the motor thresholds. The method further includes increasing magnetic stimulation pulses over subsequent treatment sessions as tolerated by the patient and obtaining repeat measures of the motor thresholds over one or both motor strips when employed for a brain disorder, or the equivalent anatomic structure when applied to an alternative region, to assess appropriate range of amplitude applied in treatment. Furthermore, the method includes repeating or maintaining the treatment as guided by patient response.

In some aspects, applying magnetic stimulation pulses in increasing amplitudes includes at least one of providing the amplitudes that rise or increase with time, providing sets of several signal trains with a same amplitude followed by a set with a higher amplitude, and providing a higher amplitude for each subsequent signal train.

The present disclosure discloses a neurophysiological magnetic stimulation system that provides repetitive magnetic stimulation in a tiered manner with each increment applied in a higher amplitude than that administered immediately prior to it. The system induces more remission of illness and larger improvements in symptoms. The system appears to be particularly effective at treating illnesses associated with decreased basal neuron activity. Specifically, the system appears to induce faster and larger response.

These and other features, aspects, and advantages of the present invention will become better understood with reference to the following description and appended claims.

### BRIEF DESCRIPTION OF THE DRAWINGS

Illustrative embodiments of the present invention are described herein with reference to the accompanying drawings, in which:

FIG. 1 depicts a block diagram of a transcranial magnetic stimulation (TMS) system in accordance with embodiments of the invention.

FIG. 2 depicts a flowchart of a TMS method in accordance with embodiments of the invention.

FIG. 3A depicts a first chart of an exemplary crescendo of amplitude of transcranial magnetic stimulation in accordance with embodiments of the invention.

FIG. 3B depicts a second chart of an exemplary crescendo of amplitude of transcranial magnetic stimulation in accordance with embodiments of the invention.

FIG. 3C depicts a third chart of an exemplary crescendo of amplitude of transcranial magnetic stimulation in accordance with embodiments of the invention.

### DETAILED DESCRIPTION OF INVENTION

For a further understanding of the nature and function of the embodiments, reference should be made to the following detailed description. Detailed descriptions of the embodiments are provided herein, as well as, the best mode of carrying out and employing the present invention. It will be readily appreciated that the embodiments are well adapted to carry out and obtain the ends and features mentioned as well as those inherent herein. It is to be understood, however, that the present invention may be embodied in various forms. Therefore, persons of ordinary skill in the art will realize that the following disclosure is illustrative only and not in any way limiting, as the specific details disclosed herein provide a basis for the claims and a representative basis for teaching to employ the present invention in virtually any appropriately detailed system, structure or manner. It should be understood that the devices, materials, methods, procedures, and techniques described herein are presently representative of various embodiments. Other embodiments of the disclosure will readily suggest themselves to such skilled persons having the benefit of this disclosure.

While the detailed description and Figures may describe the method and system as applied to a brain disorder, the invention is not limited thereto. The equivalent approach may also be employed to brainstem, spinal cord, and peripheral nervous system regions by utilizing the system and/or applying the method to the equivalent regions, anatomy, or structure of those anatomic locations and for illnesses experienced thereby.

Reference will now be made in detail to the present preferred embodiments of the invention, examples of which are illustrated in the accompanying drawings. Wherever possible, the same reference numerals are used in the drawings and the description to refer to the same or like parts.

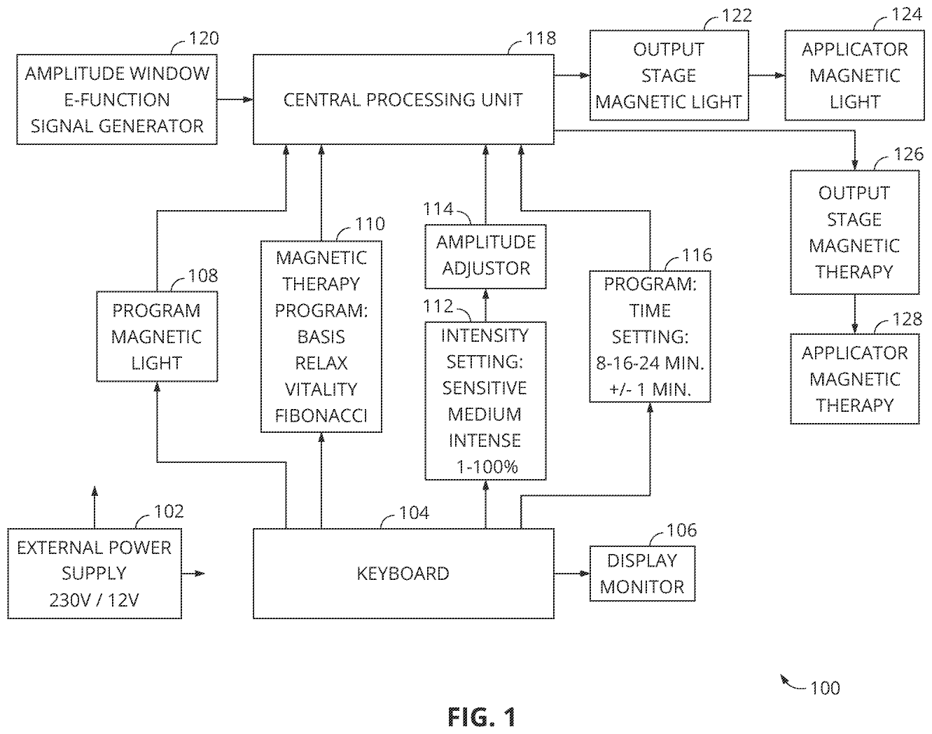

FIG. 1 depicts a block diagram of a transcranial magnetic stimulation (TMS) system 100 (or system 100) or a repetitive transcranial magnetic stimulation system, in accordance with embodiments of the invention. The system 100 may be configured to provide repetitive transcranial magnetic stimulation in a tiered manner to a patient with each increment applied in a higher amplitude than that administered immediately prior to it.

The system 100 may include a plurality of units/features including, but not limited to, a keyboard 104, a display monitor 106, a program magnetic light 108, a magnetic therapy program 110 (which may be basis, relax, vitality, or Fibonacci program), an intensity setting 112 (sensitive, medium, or intense; 1-100%), an amplitude adjustor 114, a program time setting 116 (8-16-24 minutes+/−1 minutes), a central processing unit 118, an amplitude window e-function signal generator 120, an output stage magnetic light 122, an applicator magnetic light 124, an output stage magnetic therapy 126, an applicator magnetic therapy 128, and/or the like. In some aspects, one or more these units may be powered by an external power supply 102, which may be 230V/12V power supply.

In some aspects, an operator may use the keyboard 104 to provide inputs to one or more system components or control the operation of the system components. The display monitor 106 may be configured to display system configurations/parameters, patient details and other details associated with the system and/or the patient. The program magnetic light 108 may be configured to control emission of magnetic field into the patient when, for example, the system 100 may be adhered or connected to the patient (e.g., to a patient head/brain). Specifically, the operator may “program” (or control) the emission of the magnetic field (e.g., the pulse width of the magnetic field, and/or the like) by using the program magnetic light 108.

The magnetic therapy program 110 may configured to control the supply of magnetic field to the patient (e.g., to the patient's brain), via the program magnetic light 108. In some aspects, the magnetic therapy program 110 may be configured to supply the magnetic field to the patient such that an amplitude of the magnetic field sequentially or progressively increases during the course of the treatment. The sequential increase of the magnetic field may be in a single treatment session, or over a series of multiple treatment sessions. The level of increase of the magnetic field amplitude may be programmed by the operator via the magnetic therapy program 110.

The intensity setting 112 may enable the operator to set or adjust the intensity of the magnetic field that may be fed to the patient's brain. In some aspects, the operator may adjust the intensity to medium intense. The amplitude adjustor 114 may enable the operator to set or adjust the amplitude of the magnetic field provided to the patient. As described above, the amplitude of the magnetic field may be sequentially or progressively increased during the course of the treatment. The operator may set the initial amplitude level, a maximum or final amplitude level and the sequential increase level of the magnetic field amplitude by using the amplitude adjustor 114.

The program 116 may enable the operator to set the timer for the emission of the magnetic field to the patient. For example, the operator may set the time to be 8 minutes, 16 minutes and 24 minutes (+/−1 minute) for multiple treatment sessions.

All the units described above, i.e., the program magnetic light 108, the magnetic therapy program 110, the intensity setting 112, the amplitude adjustor 114, and the time setting 116 may be controlled by inputs provided by the operator to the keyboard 104. Further, all these units may be communicatively coupled with the central processing unit 118. The central processing unit 118 may be configured to control operation of these units (and other system units) based on the inputs provided by the operator.

The central processing unit 118 may be configured to obtain signals from the amplitude window e-function signal generator 120, and control operation of the output stage magnetic light 122 and the applicator magnetic light 124 based on the signals obtained from the amplitude window e-function signal generator 120 and the inputs obtained from the operator (via the units described above). In this manner, the central processing unit 118 may perform the output stage magnetic therapy 126 and the applicator magnetic therapy 128 based on the inputs obtained from the operator (via the units described above).

A person ordinarily skilled in the art may appreciate that when employing repetitive neurophysiological magnetic stimulation (rNMS), clinicians must determine the desired parameters and target of treatment. Food and Drug Administration (FDA) approvals have thus far been for specified parameters employing fixed amplitudes maintained over the course of treatment and informed by periodic brain mapping, or equivalent anatomic site measure, events. Considerable debate persists regarding the optimal treatment parameters to employ as does the potential mechanisms responsible for rTMS' therapeutic effects. A leading hypothesis holds that high frequency rTMS induces long term potentiation of under-responsive neurons in nervous system regions pertinent to behavioral health or neurologic function. When assuming this paradigm, one may expect the amplitudes required to achieve therapeutic effects to decrease over the course of any given treatment and over the course of multiple treatments. Thus far, minimal published empiric evidence of such a decline exists.

The system 100 was used on patients by increasing amplitude of rTMS (or amplitude of magnetic field), and motor threshold (MTs) were measured over the course of rTMS. Findings from the system 100 offer meaningful commentary on the system's implications for clinical practice. The system 100 is an example of using escalating amplitude rTMS for the treatment of refractory major depression and/or other mental illnesses.

rTMS has been FDA approved for amplitudes administered at fixed ratios of the left motor strip motor threshold (LMSMT). By using the system 100 on patients, increasing amplitude treatments were employed over the course of, and within, any given treatment. The left motor strip (LMS) has played a major role in localizing the primate dorsolateral prefrontal cortex (DLPFC) and establishing the MT. Persistent strengthening of synapses based on recent patterns of activity defines long-term potentiation (LTP). If such a mechanism were responsible for the therapeutic effects of rTMS, as some contend, one may expect progressively lower amplitudes over time. Based on the test performed by using the system 100, the converse transpired. When the right motor strip motor threshold (RMSMT) was measured when using the system 100, its values were similar to that of the LMSMT at treatment initiation and unchanged following multiple treatments. Any confound at work would hence need to have raised the LMSMT, to have done so substantially, to act without impacting the RMSMT and to have done so over the timeframe the treatments were administered. Such confound would be unlikely and thus such findings are attributed to the effect of rTMS.

A person ordinarily skilled in the art may appreciate that rTMS has increasingly been applied over periods of nineteen minutes, with longer durations typically performing no better and those which are briefer worse. This may be due in part to an increase in MT over the course of a treatment, with increasingly fewer effective pulses for any given amplitude with each successive train beyond approximately minute nineteen. If this were the case, a tiered treatment, as applied using the system 100, or possibly crescendoing (or increasing) amplitudes may permit longer duration treatments to separate from that of their short duration counterparts. Conversely, among patients who find rTMS intolerable, employing a lower initial amplitude may remain a viable option, aware that subsequent treatment may be more tolerated with this technique.

A test on a patient was performed by using the system 100. The details of the test are provided below.

A 55-year-old-male with a history of major depressive disorder presented for management of depression. The patient completed self-assessment using the Patient Health Questionnaire (PHQ-9). The treatment included thirty-six sessions of high frequency (10 Hz) rTMS targeting the left dorsolateral prefrontal cortex (LDLPFC) administered as a series of daily treatments, done five times weekly. rTMS was delivered using a figure-of-8 coil connected to the system 100. A total of 3000 pulses were given as a series of 75 trains for a total of 19 minutes at an intensity of 120% of left motor strip motor threshold (LMSMT) for the initial third of treatments. Amplitudes were increased to the highest intensity tolerated for the next third of treatments and maintained during that day's treatment. The remaining third of treatments were administered such that each treatment employed a tiered amplitude. In this phase of treatment, the highest amplitude tolerated was administered with opportunity for increasing the amplitude afforded after between 25 and 38 trains followed by either a subsequent opportunity for increasing the amplitude or concluding treatment following completion of the remaining trains. Mapping was employed six times on the left and four on the right. The patient's PHQ-9 obtained prior to treatment initiation and at treatment 34 was 16 and 6 respectively. The patient's treatment amplitudes were 64% for treatments 1-14, 69-74% for treatments 15-21 and between 70-94% for treatments 22-36. His LMSMT was 53% at initiation, 65% at treatment 18, 65% and 71% immediately prior to and immediately following treatment 29 respectively and, 57% and 61% immediately prior to and immediately following treatment 34. His RMSMT was 51% and 49% immediately prior to and immediately after treatment 29 respectively, and 46% and 52% immediately prior to and immediately after treatment 34 respectively.

A person ordinarily skilled in the art may appreciate that the patient responded substantially to amplitudes gradually raised over the course of the treatment and within the course of individual rTMS treatments. Measures of MT over the left and right motor strip revealed escalating MT over the left and largely stable MTs over the right over the course of treatment. These findings raise doubts about prevailing theories on rTMS's mechanism of action as well as provide a possible framework for superior rTMS technique for the treatment of brain disorders.

The system 100, as described in the present disclosure, illustrates a novel, potentially more effective, and thus far unpublished means of utilizing rTMS for the management of major depressive disorder. Crescendoing (or increasing) amplitude rTMS appears to be associated with a therapeutic effect, increasing amplitude MT on left brain mapping, and stable amplitudes MT on right brain mapping. Test results suggest that escalating, or crescendoing, amplitude rTMS may be particularly effective for brain disorders associated with reduced basal activity, in circumstances in which a patient is poorly tolerant of conventional starting amplitude treatments, or when a patient is insufficiently responsive to orthodox, fixed amplitude, use of rTMS.

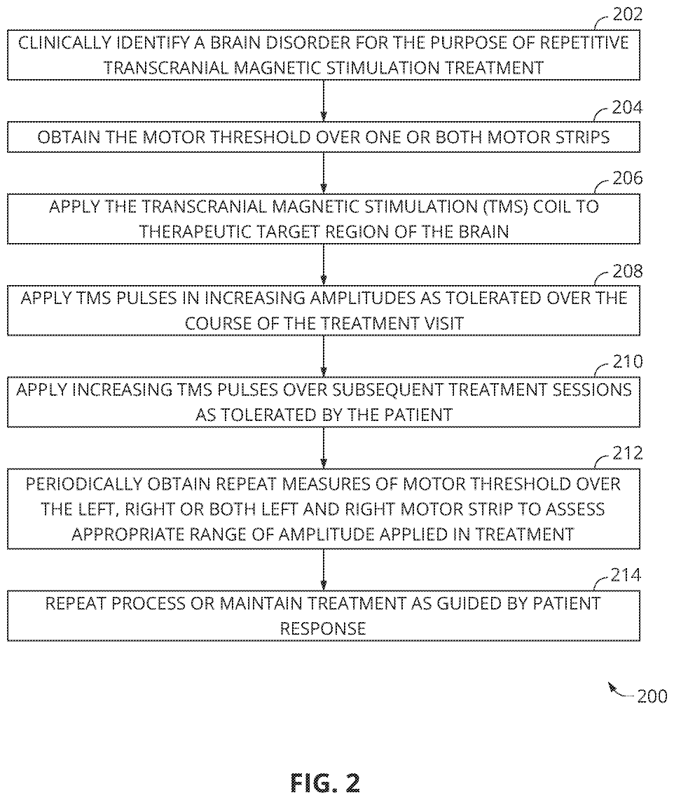

FIG. 2 depicts a flowchart of a TMS method 200 in accordance with embodiments of the invention. At step 202, the method 200 may include clinically identifying, by the central processing unit 118 (or CPU 118), a brain disorder of a patient for the purpose of repetitive transcranial magnetic stimulation treatment. At step 204, the method 200 may include obtaining, by the CPU 118, the motor threshold over one or both motor strips that may be adhered to patient's head. At step 206, the method 200 may include applying, by the CPU 118, the TMS coil associated with one or both motor strips to a therapeutic target region of the patient's brain.

At step 208, the method 200 may include applying, by the CPU 118, TMS pulses in increasing amplitudes as tolerated over the course of the treatment visit (or a treatment session). At step 210, the method 200 may include applying, by the CPU 118, increasing TMS pulses over subsequent treatment sessions as tolerated by the patient. At step 212, the method 200 may include periodically obtaining, by the CPU 118, repeat measures of motor thresholds over the left, right or both left and right motor strips to assess appropriate range of amplitude applied in treatment. At step 214, the method 200 may include repeating, by the CPU 118, the process or maintaining the treatment as guided by patient response.

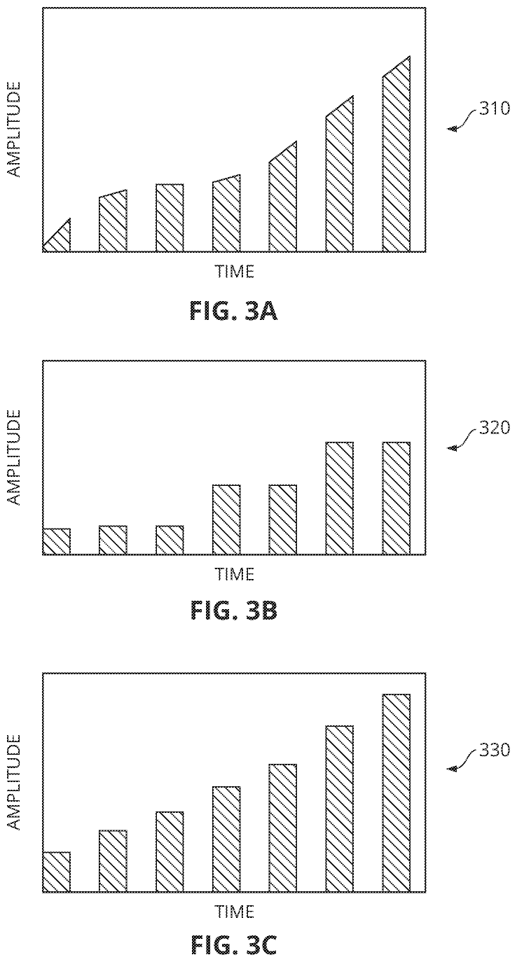

FIG. 3A depicts a first chart 310 of an exemplary crescendo of amplitude of transcranial magnetic stimulation (TMS) in accordance with embodiments of the invention. In the first chart 310, the X-axis denotes time, and the Y-axis denotes amplitude of TMS. As shown in the first chart 310, the system 100 provides the amplitudes that rise or increase with time.

FIG. 3B depicts a second chart 320 of an exemplary crescendo of amplitude of transcranial magnetic stimulation (TMS) in accordance with embodiments of the invention. In the second chart 320, the X-axis denotes time, and the Y-axis denotes amplitude of TMS. As shown in the second chart 320, the system 100 provides sets of several signal trains with the same amplitude followed by a set with a higher amplitude, followed by a set with an even higher amplitude.

FIG. 3C depicts a third chart 330 of an exemplary crescendo of amplitude of transcranial magnetic stimulation (TMS) in accordance with embodiments of the invention. In the third chart 330, the X-axis denotes time, and the Y-axis denotes amplitude of TMS. As shown in the third chart 330, the system 100 provides a higher amplitude for each subsequent signal train.

FIGS. 3A, 3B and 3C are depicted for illustrative purposes and should not be construed as limiting. Any treatment in which the amplitude of TMS is increased over the course of a treatment is consistent with the method proposed in the present disclosure.

Except as may be expressly otherwise indicated, the article “a” or “an” if and as used herein is not intended to limit, and should not be construed as limiting, the description or a claim to a single element to which the article refers. Rather, the article “a” or “an” if and as used herein is intended to cover one or more such elements, unless the text expressly indicates otherwise.

This invention is susceptible to considerable variation within the spirit and scope of the appended claims.

The claimed invention is:

1. A neurophysiological magnetic stimulation (NMS) method, comprising:
obtaining, by a central processing unit (CPU), motor thresholds over at least one motor strip adhered to a patient;
applying, by the CPU, magnetic stimulation coils of the at least one motor strip to a therapeutic target region of the patient; and
applying, by the CPU, magnetic stimulation pulses in increasing amplitudes as tolerated over a treatment session;
wherein the therapeutic target region is selected from the group consisting of brain, brainstem, spinal cord, peripheral nervous system, and any combination thereof.

2. The NMS method of claim 1, further comprising identifying a disorder of the patient before obtaining the motor thresholds.

3. The NMS method of claim 1, further comprising increasing the magnetic stimulation pulses over subsequent treatment sessions as tolerated by the patient.

4. The NMS method of claim 3, further comprising obtaining repeat measures of the motor thresholds over the at least one motor strip to assess an appropriate range of amplitude applied in a treatment.

5. The NMS method of claim 4, further comprising repeating or maintaining the treatment as guided by patient response.

6. The NMS method of claim 1, wherein applying the magnetic stimulation pulses in increasing amplitudes comprises at least one of providing the amplitudes that rise or increase with time, providing sets of several signal trains with a same amplitude followed by a set with a higher amplitude, and providing progressively higher amplitudes for each subsequent signal train.

7. A neurophysiological magnetic stimulation (NMS) method, comprising:
identifying, by a central processing unit (CPU), a disorder of a patient;
obtaining, by the CPU, motor thresholds over at least one motor strip adhered to the patient;
applying, by the CPU, magnetic stimulation coils of the at least one motor strip to a therapeutic target region of the patient; and
applying, by the CPU, magnetic stimulation pulses in increasing amplitudes as tolerated over a treatment session;
wherein the therapeutic target region is selected from the group consisting of brain, brainstem, spinal cord, peripheral nervous system, and any combination thereof.

8. The NMS method of claim 7, further comprising increasing the magnetic stimulation pulses over subsequent treatment sessions as tolerated by the patient.

9. The NMS method of claim 8, further comprising obtaining repeat measures of the motor thresholds over the at least one motor strip to assess an appropriate amplitude range applied in a treatment.

10. The NMS method of claim 9, further comprising repeating or maintaining the treatment as guided by patient response.
